(19) United States
(12) Patent Application Publication
(10) Pub. No.: US 2010/0217194 A1
(43) Pub. Date: Aug. 26, 2010

(54) Device for Tube Feeding

(76) Inventor: Ah San Pang, Singapore (SG)

Correspondence Address: COHEN, PONTANI, LIEBERMAN & PAVANE LLP, 551 FIFTH AVENUE, SUITE 1210, NEW YORK, NY, 10176, US

(21) Appl. No.: 12/086,545

(22) PCT Filed: Oct. 11, 2007
(86) PCT No.: PCT/SG2007/000346

§ 371 Date: Jun. 13, 2008

(51) Int. Cl. A61M 39/00; A61M 39/22; A61M 39/10

(52) U.S. Cl. 604/154; 604/264; 604/246; 604/174

(57) ABSTRACT

The present invention provides a device (11) for tube feeding, The device (11) comprises a delivery means (11) such as a tube for delivering food, medicines and other fluids to a patient's stomach. The delivery means (11) has a first portion (27) which is received in the patient, the first portion (27) having a maximum cross section substantially equal to or smaller than the aperture in the patient through which the first portion enters and exits so that the delivery means can be removed from the patient with minimal injury thereto, The device (11) also comprises a retaining means (19) to retain the tube in a desired position.

### FIELD OF THE INVENTION

The present invention generally relates to gastrostomy. In particular the invention relates to a device for use in percutaneous endoscopic gastrostomy.

### BACKGROUND ART

Diseases sometimes leave their victims unable to feed orally. For these patients, a tube may be used to convey liquid food to the stomach.

In those cases where the tube passes through a nostril, down the oesophagus, and into the stomach, it is known as a naso-gastric tube. When the tube passes to the stomach through the anterior abdominal wall of the patient, it is known as a gastrostomy tube. The gastrostomy tube is the preferred method when feeding via a tube is permanently required by the patient.

There are a few methods available to insert the gastrostomy tube into the patient. Of these, the pull-through percutaneous endoscopic gastrostomy (pull-through PEG) technique is the most-widely used.

Regardless of insertion technique, the gastrostomy tube must be anchored when in position to maintain the proper position. If it is not anchored the tube could migrate inwards and the tip of the tube may be caused to lie in the intestines instead of the stomach. Similarly, the tube could dislodge and detach from the patient. When the tube is dislodged, stomach contents may then leak from the patient and cause fatal peritonitis.

Currently, the gastrostomy tube is in the form of a single tube exiting the stomach through the abdominal wall. The prior art (see FIGS. 9 and 10) is in the form of a bumper and bolster combination for anchoring the tube. With this system one end of the tube has an enlarged portion, known as an internal bumper. This internal bumper is much larger that the hole through which the tube passes, preventing the tube detaching from the patient. In order to prevent inward migration, an external bolster, usually as large as the internal bumper, is designed to attach to the tube where the tube emerges from the skin. The skin of the patient and the wall of the stomach are therefore clamped between the internal bumper and the external bolster. This ensures the tube will remain in position.

The bumper does cause problems, for example ulceration and the buried bumper syndrome. It also becomes an obstacle when the gastrostomy tube has to be changed percutaneously. Every tube will require changing after a period of use because of wear and tear, or blockage. In order to replace the tube it is first necessary to remove the tube before a new tube can be inserted.

When removing the tube anchored using a bolster and bumper combination a fair amount of force is needed to pull the large bumper through the small hole. Consequently, traumatic bleeding is inevitable.

It is common for the patient to pull out the gastrostomy tube either accidentally or deliberately and the care-givers remain unaware until several hours later when severe problems arise. Another common situation is the care-givers accidentally pulling out the gastrostomy tube when they turn, move, bathe, or dress the patient (often comatose and bed-ridden).

To minimize trauma, the bumper can be made less rigid so that the tube can be pulled out with less force. However, a flexible or collapsible bumper is associated with a higher frequency of accidental dislodgement of the tube, a problem which the bumper was meant to prevent in the first place.

The preceding discussion of the background to the invention is intended only to facilitate an understanding of the present invention. It should be appreciated that the discussion is not an acknowledgment or admission that any of the material referred to was part of the common general knowledge as at the priority date of the application.

It is an object of this invention to provide a device for tube feeding having a more robust retaining means which permits a less traumatic change of the gastrostomy tube.

### SUMMARY OF INVENTION

The present invention provides a device for tube feeding, the device comprises a delivery means for delivering food, medicines and other fluids to a patient's stomach, the delivery means having a first portion which is received in the patient, the first portion having a maximum cross section substantially equal to or smaller than the aperture in the patient through which the first portion enters and exits so that the delivery means can be removed from the patient with minimal injury thereto, the device also comprises a retaining means to retain the tube in a desired position.

In contrast to the prior art, the current invention can be removed from the patient without causing trauma thereto. As the cross section of the first portion of the tube which enters the patient is the same or smaller than the aperture in the patient the delivery means will not require a larger aperture in order to allow the delivery means to be removed.

Preferably the delivery means is in the form of a tube.

The tube may enter the patient through a first aperture in the patient, pass through the patient's stomach and exit the patient through a second aperture.

In one aspect of the invention the external diameter of the first portion of the tube is uniform.

In another aspect the first portion may have varying diameters wherein the largest diameter is substantially equal to or smaller than the aperture in the patient.

The tube may be made from a flexible, clear material.

Preferably the internal diameter of the tube remains substantially the same when curved or looped.

The first portion may incorporate an exit means for the fluid to leave the tube and enter the stomach. Preferably the exit means is in the form of at least one opening. In use the opening is positioned in the stomach of the patient so that the fluid delivered to the patient enters the stomach.

The at least one opening may be elongate in shape. The at least one opening may be elliptical.

The at least one opening may be larger than the internal diameter of the tube. This will ensure that the opening does not inhibit the flow of fluid to the stomach.

The tube may have a plurality of markings along the length of the tube. This will allow a medical professional to judge the position of the opening when placing the tube in the patient to ensure the opening is aligned with the stomach. The markings also provide an indication as to which side the at least one hole is located.

Preferably each end of the tube is the same and is adapted to allow the tube to be pulled through the patient (PEG insertion). The tube may be adapted for insertion by techniques other than PEG insertion.

The tube may incorporate a solid portion.

The tube may be reinforced with metal.

The tube may have two openings wherein a wall in the tube separates the openings. This will allow different fluids to be delivered to the stomach, as may be required with slow release medicines.

The tube may be moulded so as to provide the desired shape: U shaped, loop or circular.

Each open end of the tube may have a valve attached thereto to prevent reflux of stomach contents.

The retaining means may be in the form of a clamp capable of moving from an open position to a closed position, the clamp being adapted to retain a second portion and a third portion of the delivery means relative to each other, whereby the first portion is located between the second portion and third portion. When the second portion and third portion are clamped the tube extending between the portions may be in a circular form.

The clamp may receive the second portion and third portion such that they are retained in a side by side relationship.

When the clamp is in the closed position the second portion and third portion may be clamped relative to each other. Preferably a key is required to open the clamp. This will ensure the clamp is not erroneously opened.

Preferably when the clamp is closed and the tube retained therein the at least one opening is facing away from the clamp.

The retaining means may incorporate an alarm means which is activated when the retaining means detects any force which may be accidentally applied to pull apart the system.

The device may also comprise a connector wherein one end of the connector is adapted to be inserted in an end of a first tube and the second end of the connector is adapted to be inserted in an end of a second tube, the connector being of equal or smaller diameter than the external diameter of the first tube and/or second tube. The connector may be used to connect a tube already in position in a patient with a replacement tube such that the tube may be replaced with a new tube with minimal impact upon the patient.

The device may also comprise one or more of the following: a universal port for delivery systems, a sensor/alarm which sounds when the tube is detached, a flow regulator and/or a drug chamber.

The retaining means may incorporate an infusion pump for the slow delivery of feeds, and/or a sterilizing apparatus to sterilize the food before it passes into the stomach.

The retaining means may incorporate a connector to allow a delivery system to be connected to the device to allow food and other medicines to be delivered to the stomach.

### BRIEF DESCRIPTION OF THE DRAWINGS

The invention will be better understood by reference to the following description of several specific embodiments thereof as shown in the accompanying drawings in which:

FIG. 1 is an illustration of the device according to a first embodiment of the invention;

FIGS. 2a, b, c are illustrations of the retaining means in various positions;

FIG. 2d is an illustration of a key;

FIG. 3a is an illustration of the device in position in a first arrangement;

FIG. 3b is an illustration of the device in position in a second arrangement;

FIG. 4 is an illustration of a device according to a second embodiment and in position in a third arrangement (a vestigial arrangement);

FIG. 5 is an illustration of a device according to a third embodiment wherein the retaining means incorporates a delivery system and/or a sensor/alarm system;

FIG. 6 is an illustration of a device according to a fourth embodiment wherein the retaining means incorporates a sterilizing portion and/or a sensor/alarm system;

FIG. 7 is an illustration of a connector;

FIG. 8 is an illustration of a valve;

FIG. 9 is an illustration of a prior art device; and

FIG. 10 is an illustration of FIG. 9 in position.

### BEST MODE(S) FOR CARRYING OUT THE INVENTION

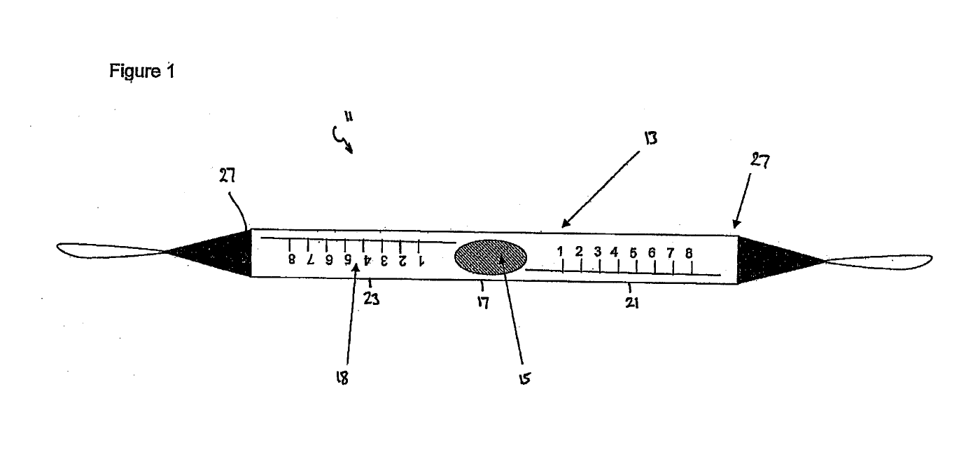

Referring to FIG. 1, the invention according to the first embodiment is in the form of a device 11 for tube feeding. The device 11 comprises a delivery means in the form of a tube 13.

The tube 13 is made from a soft flexible medical-grade silicone or polymer (such as polypropylene, polyurethane or other material suitable for use as a gastrostomy tube) whereby the tube 13 can form a loop without kinking, or narrowing of the internal diameter.

The tube 13 incorporates an exit means which is in the form of an opening 15 located in a first portion 17 of the tube 13. The opening 15 is elliptical in shape. This shape will assist in maintaining the integrity of the tube 13 so that the tube 13 does not break into two pieces.

The opening 15 has a diameter greater than the internal diameter of the tube 13 to ensure the opening 15 does not inhibit the flow of fluid to the stomach.

The tube 13 also has two identical sets of markings 18. Each set of markings extends either side of the opening 15 towards opposite ends of the tube 13.

Each end of the tube 13 has a fitting 27 connected thereto. The fittings 27 enable the tube to be pulled through a patient.

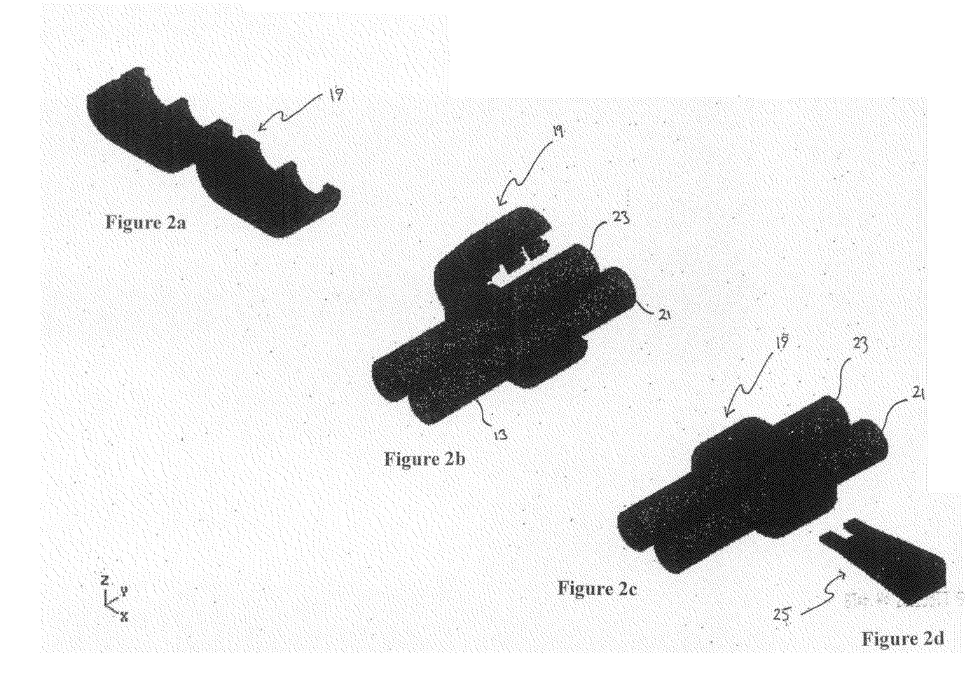

Referring to FIGS. 2a, b, and c, the device 11 also comprises a retaining means in the form of a clamp 19. The clamp is movable between an open position, as shown in FIG. 2a, and a closed position, as shown in FIG. 2c. The clamp is adapted to receive a second portion 21 and a third portion 23 of the tube 13 in side by side relationship. The second portion 21 and third portion 23 are positioned either side of the first portion 17, as shown in FIGS. 2b and 2c.

When in the closed position the clamp 19 is latched firmly closed so that the clamp 19 can not accidentally open. In order to open the clamp 19 a key 25 may be used, as shown in FIG. 2d.

When placing the tube 13 in a patient using the pull-through PEG technique, one end of the tube passes through the stomach 30 and exits the patient through a first aperture 29, and the other end similarly exits through a second aperture 31. Using the markings 18, a medical professional positions the opening 15 such that it is in the patient's stomach 30. Again using the markings 18 the tube 13 is orientated so that the opening 15 faces outwardly within the patient.

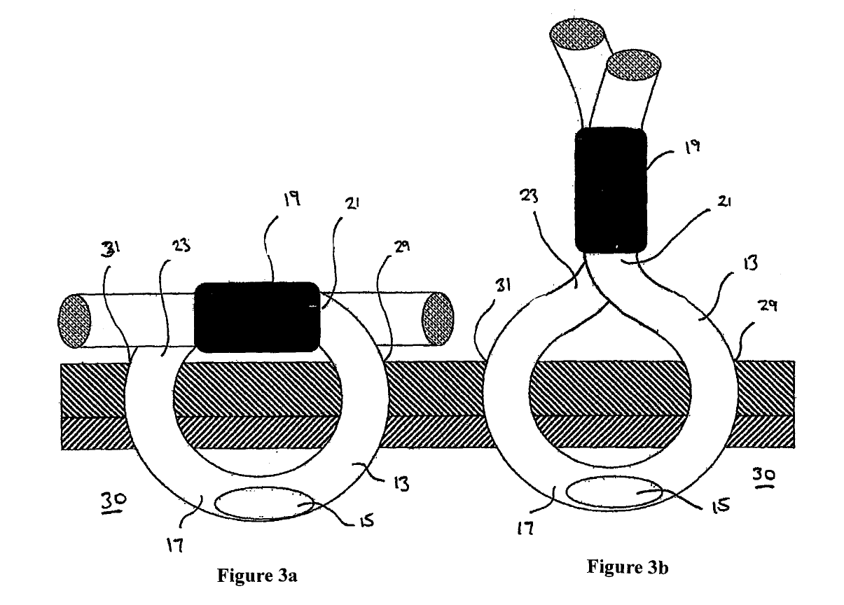

Once in position it is important that the tube 13 be retained in position. In order to achieve this the second portion 21 and third portion 23 are crossed and retained with respect to each other using the clamp 19 such that they are in side by side relationship. As shown in FIG. 3a the tube 13 may be arranged so that the ends of the tube 13 extend in opposite directions when leaving the clamp 19, or, as shown in FIG. 3b the ends may extend in the same direction as each other.

When properly retained, the clamp 19 is located between the first aperture 29 and second aperture 31 of the patient, which also corresponds to the positioning of the opening 15 of the tube 13 in the stomach.

When the clamp 19 is in position the tube 13 can not move inwardly with respect to the patient nor can it be dislodged.

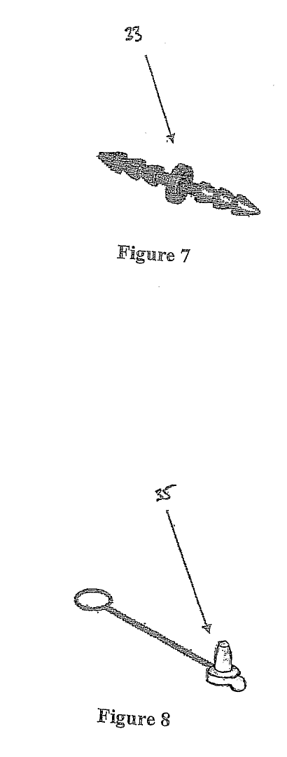

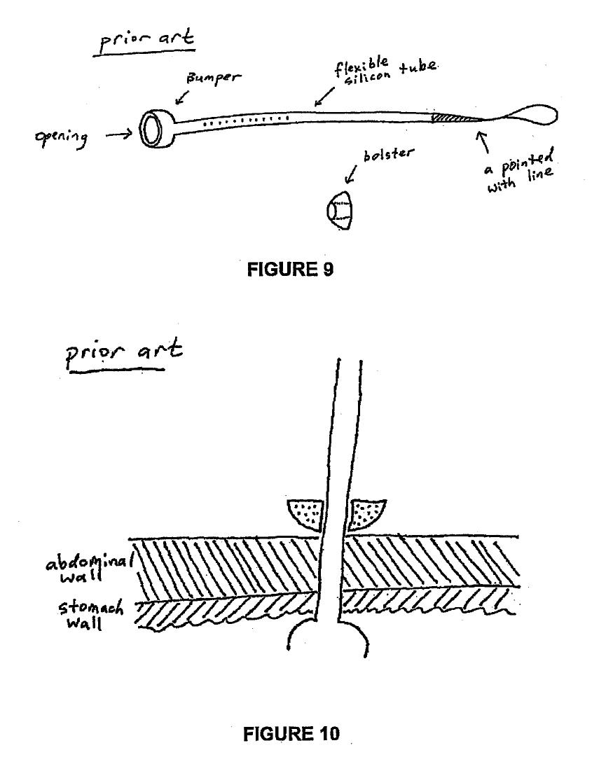

At times it may be necessary to replace the tube 13 in a patient's stomach with a new one. With the current invention this is a simple task. For this purpose the device 11 comprises a connector 33, as shown in FIG. 7. In use, the connector 33 is fitted to an end of a first tube 13a, and an end of a second tube 13b. As the first tube 13a is removed from the stomach, the second tube 13b is pulled into position as it follows the first tube 13a. When the second tube 13b is in the desired position the first tube 13a can be removed and the clamp 19 fitted to the second tube 13b.

The device 11 also comprises valves 35, as shown in FIG. 8, to prevent reflux of the stomach contents.

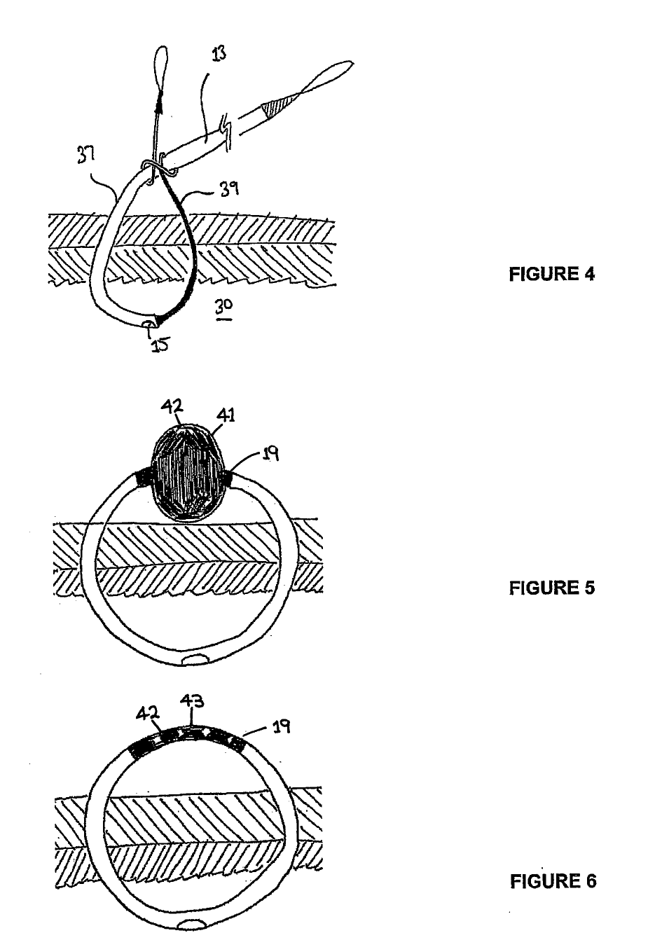

According to another embodiment of the invention, the tube 13 may have a hollow portion 37 and an elongate, solid (vestigial) portion 39, as shown in FIG. 4. In this embodiment, the tube 13 may be arranged such that the retaining means is formed by the tube 13 whereby the elongate portion 39 is tied with respect to the hollow portion 37 so that the two portions are retained with respect to each other and the device remains in position. Clamp 19 may be used to reinforce the tie.

In other embodiments the tube 13 may have sections of different external diameter. In these arrangements it is important that when changing the tube 13 the smaller diameter portion is used as the leader. A suture is tied to the smaller diameter portion and pulled out through both exit openings. The suture is then used to rail-road the replacement tube, with the smaller diameter portion of the new tube going in through the larger exit opening first.

FIG. 5 shows a third embodiment of the invention wherein the clamp 19 is configured to incorporate a delivery system 41 such as those which use an infusion pump for slow delivery of feeds and/or a sensor/alarm system 42 to detect if the device has been moved or dislodged.

FIG. 6 shows a fourth embodiment of the invention wherein the clamp 19 is configured to incorporate a sterilizing portion with a UV-light source 43 and/or a sensor/alarm system 42 to detect if the device has been moved or dislodged.

The tube 13 may have a radio-opaque stripe to make it visible to X-ray. The tube may be coated with eluding antibiotics or other drugs.

Whilst the invention is described in relation to one particular application it is understood that other applications are also protected by this invention. For instance, the device could be employed as a feeding jejunostomy, a tube sigmoid colostomy, a suprapubic cystostomy, and a pelvic, peritoneal, or thoracic drain.

Modifications and variations such as would be apparent to the skilled addressee are considered to fall within the scope of the present invention.

Throughout the specification, unless the context requires otherwise, the word “comprise” or variations such as “comprises” or “comprising”, will be understood to imply the inclusion of a stated integer or group of integers but not the exclusion of any other integer or group of integers.

1. A device for tube feeding, comprising a delivery means for delivering food, medicines and other fluids to a patient, the delivery means having a maximum cross section substantially equal to or smaller than the aperture in the patient through which the delivery means enters and exits, wherein the delivery means is adapted to enter the patient through a first aperture in the patient, pass through the patient and exit the patient through a second aperture.

2. The device of claim 1 wherein the delivery means is a tube.

3. The device of claim 1 further comprising a retaining means to retain the tube in a desired position.

4. The device of claim 1 wherein the external diameter of the delivery means is uniform.

5. The device of claim 1 comprising a first portion, the first portion varies in diameter to other portions of the delivery means wherein the first portion being the largest diameter is substantially equal to the first aperture and/or the second aperture in the patient.

6. The device of claim 2 wherein the tube is made from a flexible, clear material.

7. The device of claim 2 wherein the internal diameter of the tube remains substantially the same when curved or looped.

8. The device of claim 1 wherein the delivery means incorporates an exit means for the fluid to leave the tube and enter the patient.

9. The device of claim 8 wherein the exit means is in the form of at least one opening.

10. The device of claim 1 wherein the delivery means is adapted to enter the patient through the first aperture in the patient, pass through the patient's stomach, or jejunum, or colon, or, urinary bladder, or pelvis, or abdomen, or thorax and exit the patient through a second aperture.

11. The device of claim 9 wherein the at least one opening is elliptical.

12. The device of claim 9 wherein the at least one opening is larger than the internal diameter of the tube.

13. The device of claim 1 wherein the delivery means has a plurality of markings along the length of the tube.

14. The device of claim 2 wherein each end of the tube is the same and is adapted to allow the tube to be pulled through the patient.

15. The device of claim 2 wherein the tube incorporates a solid portion.

16. The device of claim 2 wherein the tube is reinforced with metal.

17. The device of claim 2 wherein the tube has two openings and wherein a wall in the tube separates the two openings.

18. The device of claim 2 wherein the tube is molded to provide a desired shape such as U shaped, loop or circular.

19. The device of claim 2 wherein the opening of the tube comprises a valve for prevention of reflux of stomach contents.

20. The device of claim 3 wherein the retaining means comprises a clamp capable of moving from an open position to a closed position, the clamp being adapted to retain a second portion and a third portion of the delivery means relative to each other.

21. The device according to claim 20 wherein when the second portion and third portion are clamped the delivery means extending between the portions is in a circular form.

22. The device of claim 20 wherein the clamp receives the second portion and third portion such that they are retained in a side by side relationship.

23. The device of claim 20 wherein when the clamp is in the closed position the second portion and third portion may be clamped relative to each other.

24. The device of claim 20 wherein a key is required to open the clamp.

25. The device of claim 20 wherein at least one opening is facing away from the clamp when the clamp is in the closed position.

26. The device of claim 3 wherein the retaining means comprises an alarm means which is activated when the retaining means detects any force which may be accidentally applied to pull apart the system.

27. The device of claim 1 comprising a connector wherein one end of the connector is adapted to be inserted in an end of a first tube and the second end of the connector is adapted to be inserted in an end of a second tube, the connector being of equal or smaller diameter than the external diameter of the first tube and/or second tube such that the first tube already in position in a patient is connected to the second tube the second tube is pulled through the apertures in the patient to replace the first tube with minimal impact upon the patient.

28. The device of claim 1 further comprising one or more of the following: a universal port for delivery systems, a sensor/alarm which sounds when the tube is detached, a flow regulator and/or a drug chamber.

29. The device of claim 3 wherein the retaining means further comprises an infusion pump for the slow delivery of feeds.

30. The device of claim 3 wherein the retaining means further comprises a sterilizing apparatus to sterilize the food before it passes into the patient.

31. The device of claim 3 wherein the retaining means further comprises a connector to allow a delivery system to be connected to the device to allow food and other medicines to be delivered to a patient.

32. The device of claim 2 wherein the tube is employed as a drain.

33. (canceled)
